(19) United States
(12) Patent Application Publication
(10) Pub. No.: US 2026/0166192 A1
(43) Pub. Date: Jun. 18, 2026

(54) Sterilization Indicator Sensor

(71) Applicant: Solventum Intellectual Properties Company, Maplewood, MN (US)

(72) Inventors: Wensheng Xia, Woodbury, MN (US); Naiyong Jing, St. Paul, MN (US)

(21) Appl. No.: 18/707,803

(22) PCT Filed: Oct. 12, 2022
(86) PCT No.: PCT/IB2022/059787

§ 371 Date: May 6, 2024

Related U.S. Application Data
(60) Provisional application No. 63/263,893, filed on Nov. 11, 2021.

(51) Int. Cl. A61L 2/28; A61L 2/07; A61L 103/15; G01N 27/12

(52) U.S. Cl. CPC A61L 2/28; A61L 2/07; G01N 27/121; A61L 2103/15; A61L 2202/122; A61L 2202/14

(57) ABSTRACT

In some embodiments, a sensor device includes a first electrode and a second electrode, each of the first and second electrodes being electrically coupled to an electrical bridge. The electrical bridge includes a conductive polymer having a first impedance state and a second impedance, the second impedance state being different than the first impedance state. The sensor device also includes a latent base.

### BACKGROUND

Various chemical indicators for sterilization monitoring are described in, for example, U.S. Pat. App. Pub. 2012/0100395, U.S. Pat. Nos. 3,523,011 and 5,064,576.

### BRIEF DESCRIPTION OF THE DRAWINGS

The disclosure may be more completely understood in consideration of the following detailed description of various embodiments of the disclosure in connection with the accompanying figures, in which:

FIG. 1 illustrates a sterilization system that can be used in connection with the sensors of the present disclosure.

FIG. 2 illustrates a sensor device in accordance with some embodiments of the present disclosure.

FIG. 3 illustrates use of a sensor device in a sterilization system in accordance with some embodiments of the present disclosure.

FIG. 4 illustrates use of a sensor device in a sterilization system in accordance with some embodiments of the present disclosure.

While the above-identified drawings, which may not be drawn to scale, set forth various embodiments of the present disclosure, other embodiments are also contemplated, as noted in the Detailed Description. In all cases, this disclosure describes by way of representation of exemplary embodiments and not by express limitations. It should be understood that numerous other modifications and embodiments can be devised by those skilled in the art, which fall within the scope and spirit of this disclosure.

### DETAILED DESCRIPTION

Chemical indicators are widely used in sterilization monitoring of medical devices (e.g., surgical instruments) to ensure the sterilization process has been completed correctly. Failed or insufficient sterilization cycles pose significant patient risk due to potential cross-contaminations from the reprocessed medical devices.

Traditional chemical indicators are based on colorimetric changes in the presence of a certain sterilant and its running conditions such as sterilization temperature or sterilization time. For example, a steam indicator may change color from light yellow to black. In the current practice of evaluating a chemical indicator visually, a user needs to visually judge the color development to determine if the chemical indicator was subjected to an adequate sterilization process. However, color development can be subjective. As a result, articles and methods for more objectively determining the adequacy of sterilization processes are desirable.

For the following defined terms, these definitions shall be applied for the entire Specification, including the claims, unless a different definition is provided in the claims or elsewhere in the Specification based upon a specific reference to a modification of a term used in the following definitions:

The terms “about” or “approximately” with reference to a numerical value or a shape means +/−five percent of the numerical value or property or characteristic, but also expressly includes any narrow range within the +/−five percent of the numerical value or property or characteristic as well as the exact numerical value. For example, a temperature of “about” 100° C. refers to a temperature from 95° C. to 105° C., but also expressly includes any narrower range of temperature or even a single temperature within that range, including, for example, a temperature of exactly 100° C. For example, a viscosity of “about” 1 Pa-sec refers to a viscosity from 0.95 to 1.05 Pa-sec, but also expressly includes a viscosity of exactly 1 Pa-sec. Similarly, a perimeter that is “substantially square” is intended to describe a geometric shape having four lateral edges in which each lateral edge has a length which is from 95% to 105% of the length of any other lateral edge, but which also includes a geometric shape in which each lateral edge has exactly the same length.

The terms “a”, “an”, and “the” include plural referents unless the content clearly dictates otherwise. Thus, for example, reference to a material containing “a compound” includes a mixture of two or more compounds.

“Latent base” refers to a compound or composition which, when incorporated into another composition, can act as a controlled (e.g., in response to an environment condition) release of base into said composition. That is, depending on, for example, environmental conditions (e.g., temperature or temperature and exposure to moisture), the compound or composition may or may not function as a base.

“Base” refers to a compounds or composition acting as an electron pair donor, exemplified by Lewis bases or Bronsted bases.

“Conductive element” refers to refers to an ability to conduct an electric current. Electrically conductive materials have an electrical conductivity of at least 2 Siemens per centimeter. Exemplary conductive elements include silver, gold, copper, aluminum, or combinations thereof.

“Adequate sterilization process” refers to a sterilization process that achieves a sterility assurance level of 10⁻⁶, or 12 log reduction of Bacillus Subtilis var. Niger. The sterility assurance level is related to a probability that a sterilized unit remains nonsterile after undergoing the sterilization process.

“Adequate environmental condition” refers to environmental conditions inside of a sterilization chamber that correspond to the adequate sterilization process.

The phrase “comprises at least one of” followed by a list refers to comprising any one of the items in the list and any combination of two or more items in the list. The phrase “at least one of” followed by a list refers to any one of the items in the list or any combination of two or more items in the list.

Although the term “impedance” is used, the term “impedance” is the reciprocal of the “admittance”. Depending on the context, either impedance or admittance can be used as changes in the impedance of a material.

The term “and/or” means one or all of the listed elements or a combination of any two or more of the listed elements.

All numerical ranges are inclusive of their endpoints and nonintegral values between the endpoints unless otherwise stated (e.g., 1 to 5 includes 1, 1.5, 2, 2.75, 3, 3.80, 4, 5, etc.).

Before any embodiments of the present disclosure are explained in detail, it is to be understood that the present disclosure is not limited in its application to the details of use, construction, and the arrangement of components set forth below. The present disclosure is capable of other embodiments and of being practiced or of being carried out in various ways that will be apparent to a person of ordinary skill in the art. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

Unless otherwise indicated, all numbers expressing quantities or ingredients, measurement of properties and so forth used in the Specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

In some embodiments, the present disclosure relates to a sterilization system and associated sensor device having a sterilant-responsive switch that may be responsive to environmental conditions (including the presence of a sterilant such as steam) in a sterilization process. Generally, the sensor devices of the present disclosure enable electronical reporting of information (e.g., pass/fail information, accept/reject information) regarding each sterilization cycle to avoid subjective judgements that can lead to errors (e.g., perceived change in color by the human eye). Also, the systems and devices of the present disclosure enable digitalization of sterilization results which, in turn, will free technicians from manual document and physical storage.

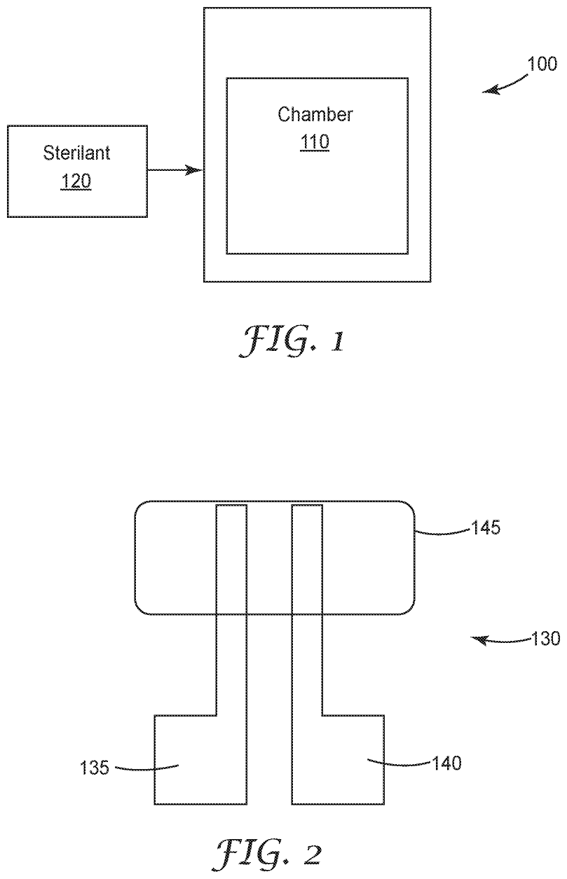

FIG. 1 illustrates a sterilization system 100 in which a sensor device of the present disclosure may be employed. As shown in FIG. 1, the sterilization system 100 may include a chamber 110 into which a sterilant stream 120 may be directed. The sterilization system 100 may be of a type commonly used by hospitals and other medical facilities to sterilize reusable medical devices. Various types of sterilization systems 100 can be employed for purposes of the present disclosure. For example, the sterilization systems 100 can be based on steam or hydrogen peroxide (e.g., vaporized hydrogen peroxide), and each type can have different sterilization process conditions. Examples of sterilizer systems using hydrogen peroxide as a sterilant are commercially available from Steris (Mentor, OH) or Tuttnauer (Israel). Examples of sterilizers using steam as a sterilant are commercially available from Steris (Mentor, OH) or Getinge (Gothenburg, Sweden).

In some embodiments, the chamber 110 can have one or more environmental conditions. The environmental conditions can be related to conditions inside of the chamber 110 and can include, for example, exposure time, sterilant (presence, concentration, etc.), temperature, pressure, or combinations thereof. In some embodiments, a first environmental condition can exist pre-sterilization process and a second environmental condition can exist during the sterilization process.

In some embodiments, the present disclosure is directed to a sensor device that is configured to determine whether a sterilization process within a sterilization system is carried out in accordance with a predetermined guideline or whether an adequate sterilization process was achieved. An adequate sterilization process can vary based on the sterilant used, the manufacturer of the sterilizer, or the articles to be sterilized. For example, Guideline for Disinfection and Sterilization in Healthcare Facilities, Center for Disease Control (2008), which is herein incorporated by reference in its entirety, provides minimum cycle times for sterilization of various article types and sterilants.

Referring to FIG. 2, a sensor device 130 in accordance with some embodiments of the present disclosure is depicted. The sensor device 130 may include a first electrode 135, a second electrode 140 (sometimes, collectively, referred to as an electrode pair), and a sterilant-responsive electrical bridge 145 which may facilitate electrical communication between the first electrode 135 and the second electrode 140. In some embodiments, each of the first electrode 135 and the second electrode 140 may be in electrical communication, or electrically coupled, (either via physical contact or via an intermediate such as a conductive member (e.g., an electrically conductive wire)) via the sterilant-responsive electrical bridge 145. As shown in FIG. 2, in some embodiments, an end of each of the first electrode 135 and the second electrode 140 may be in physical contact with the sterilant-responsive electrical bridge 145. In some embodiments, absent the sterilant-responsive electrical bridge 145, the electrode pair 135, 140 may not be capable of electrical communication (i.e., the electrodes are not physically touching or are spaced apart at least a distance such that there is no electrical communication without an intervening conductive member).

In some embodiments, the first and second electrodes 135, 140 may include a metal such as aluminum, iron, zinc, tungsten, molybdenum, tin, nickel, copper, or alloys thereof, or carbon black, graphene, carbon nanotubes, or a conducting polymer.

In some embodiments, the electrical bridge 145 may be configured to have a first impedance state (e.g., high impedance/no or low conductivity) and a second impedance state that is markedly different than the first impedance state (e.g., low impedance/high conductivity (or vice versa). For example, in some embodiments, in a first state, the electrical bridge exhibits a low impedance and in a second state exhibits a high impedance (relative to the low impedance state). In some alternative embodiments, in a first state, the electrical bridge exhibits a low electric capacitance and in a second state exhibits a high electric capacitance (relative to the low electric capacitance state) or vice versa.

In some embodiments, referring still to FIG. 2, the electrical bridge 145 may include a conductive polymer and a latent base. For example, the conductive polymer and latent base may be dispersed in a polymeric binder and deposited onto the electrode pair. As another example, the conducting polymer may be disposed in a layer that is deposited on the electrode pair (without latent base) and the latent base may be present in a sterilant soluble (e.g., steam soluble) layer that is coated on the conductive polymer layer such that the latent base will diffuse into the conducting polymer after exposure to the sterilant.

Generally, the conductive polymer material can be any polymeric material that may be shifted between a first impedance state and a second impedance state. In some embodiments, suitable conductive polymers may be those capable of being converted a first impedance state to a second impedance state in response to a change of environmental conditions (e.g., transitioning from the first state to the second state upon contact with a sterilant, or transitioning from the first state to the second state upon achievement of an adequate sterilization process within a sterilizer system). In some embodiments, the first state can be a low impedance state and the second state can be a high impedance state (or vice versa). In some embodiments, the low impedance state can be a doped (e.g., acid doped) electrically conductive state and the high impedance state can be can be a de-doped (e.g., by inclusion and activation of a basic material) electrically non-conductive (or at least a conductivity lower than that of the electrically conductive state). In some embodiments, a low impedance state refers to a state having an admittance sufficient to electrically bridge an open circuit, e.g., having an admittance of at least 2 siemens.

In some embodiments, the conductive polymer material of the electrical bridge 145 can have a repeat unit of: aniline, acetylene, pyrrole, phenylene, phenylene vinylene, phenylene ethynylene, phenylene sulfide, fluorene, pyrene, azulene, naphthalene, carbazole, indole, thiophene, ethylene dioxythiophene, or combinations thereof. The conductive polymer material can be doped or undoped with various dopants such as dinonylnaphthalene sulfonic acid (DNNSA), dodecylbenzenesulfonic acid (DBSA), arsenic pentafluoride, triiodide, camphorsulfonate, methanesulfonic acid, halogens or polyhalogen ions, methanol, hydrogen sulfate, hydrochloric acid, tetrafluoroborate, sodium sulfite, or combinations thereof.

In some embodiments, the conductive polymer material includes (or consists essentially of) polyaniline (PANI). In some embodiments, the conductive PANI is in a form of electrolytes polyelectrolytes or PANI salts which can be readily achieved by acid-doping of PANI. PANI can be in one of three oxidation states (leucoemeraldine, emeraldine (in the salt or base forms), and per (nigraniline)). The emeraldine can be non-conductive in the base form and conductive in the polyelectrolyte form or the salt form. The emeraldine salt can be converted into the leucoemeraldine salt or per (nigraniline) which are non-conductive, via a redox reaction. The conductive polymer can be converted to non-conductive polymer via a de-doping reaction. In some embodiments, the conductive polymer material of the present disclosure may be present, initially, in the conductive emeraldine salt form (acid form) and be convertible to the non-conducting emeraldine form (base form) upon exposure to a sterilant.

In some embodiments, the latent base may be any known compound or composition capable of controlled release of its functionality as a base. For example, the latent base may be any compound or composition capable of release of it functionality as a base in response to a change in environmental condition (e.g., temperature, pressure, exposure to a particular material (e.g., steam), exposure to light, or combinations thereof). In some embodiments, the latent bases may include compounds that are poorly soluble or insoluble in water at room temperature but that are more soluble in water at elevated temperatures (e.g., greater than 80 degrees Celsius). Consequently, when exposed to steam, such a latent base will be released into the conductive polymer. For purposes of the present disclosure a latent base's release of its functionality as a base may be referred to as activation of the latent base.

In some embodiments, useful latent bases are those that can release active bases exemplified by aluminum hydroxide or other metal hydroxide (see below) upon exposure to a sterilant (e.g., steam). For example, suitable latent bases include those that can be activated to capture protons to neutralize PANI electrolytes or polyelectrolytes (protonated forms) to its neutral or less protonated emeraldine form. Examples of such latent bases include

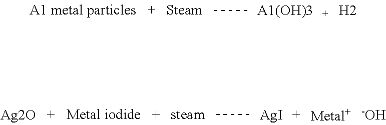

In a similar fashion, certain organic bases may be suitable latent bases. For example, organic bases that may be activated by a sterilant (e.g., steam) via Hoffman elimination may be employed. Hofmann elimination is an elimination reaction of an amine where the least stable (least substituted) alkene, the Hofmann product, is formed. This tendency, known as the Hofmann alkene synthesis rule, is in contrast to usual elimination reactions, where Zaitsev's rule predicts the formation of the most stable alkene. The reaction involves the formation of a quaternary ammonium iodide salt by treatment of the amine with excess methyl iodide (exhaustive methylation), followed by treatment with silver oxide and water to form a quaternary ammonium hydroxide. When this salt is decomposed by heat, the Hofmann product is preferentially formed due to the steric bulk of the leaving group causing the hydroxide to abstract the more easily accessible hydrogen, as shown in the flow diagram below.

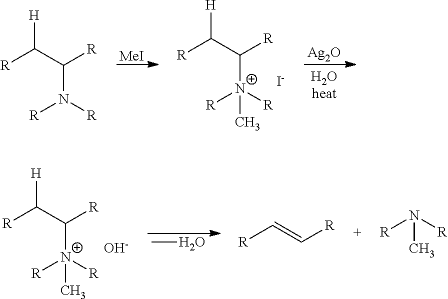

In some embodiments, suitable latent bases may include metal carbonates (e.g., lithium carbonate, lead carbonate, calcium carbonate, strontium carbonate, barium carbonate, zinc carbonate, etc.), or metal hydroxides (e.g. strontium hydroxide, barium hydroxide, zinc hydroxide, etc.) metal sulfide compounds (e.g. calcium sulfide, etc.), metal complexes (e.g. zirconium chloranilate), an exchange ligand (e.g. citric or tartaric acid salts and amino carboxylic acid), or a mixture of (a) 2,4-dihydroxybenzoic acid and its metal salt and (b) phenylpropionic acid and its metal salt.

In some embodiment, the suitable latent bases may have a pKb value that is equal to or larger than that of PANI emeraldine form.

In embodiments that include an polymeric binder, the polymeric binder can include any suitable polymeric binder, for example, a polyurethane, a polyvinyl butyral, a polyacrylate, polyvinyl acetate, polystyrene, polystyrene acrylate, a polyurea, a polyimide, an amide, an epoxy, a glycidyl-Si—Zr-containing solgel, a polyester, a phenoxy resin, a polysulfide, or mixtures thereof.

In some embodiments, conductive polymer may be present in the electrical bridge 45 in an amount of at least 5 wt. %, at least 10 wt. %, or at least 50 wt. %, based on the total weight of the composite material that forms the electrical bridge 45.

In some embodiments, latent base may be present in the electrical bridge 45 in an amount of at least 0.01 wt. %, at least 0.1 wt. %, or at least 20 wt. %, based on the total weight of the composite material that forms the electrical bridge 145. Generally, the amount of latent base present in the electrical bridge may be that which is necessary to convert the conductive polymer from the acid state to the dedoped state upon release of the latent base.

In some embodiments, in addition to a change in impedance state, the electrical bridge 145 may additionally exhibit a change in color. For example, in embodiments in which the electrical bridge 145 includes PANI, the electrical bridge 145 may begin in a first impedance state (e.g., acid doped) having a green color and a second impedance state (e.g., de-doped) having a blue color. In this manner, visual determination of the adequacy of a sterilization cycle may be carried out.

In some embodiments, the sensor device 130 may be a stand-alone device that can be placed into a sterilization system 100. In further embodiments, the sensor device 130 may be incorporated into another device (e.g., sterilization process challenge device with a torturous path such as porous matrix or a lumen channel, Bowie-Dick test pack, or the like) which may include a housing and one or more internal components or materials that are configured to facilitate assurance that adequate sterilization conditions are present during a sterilization cycle.

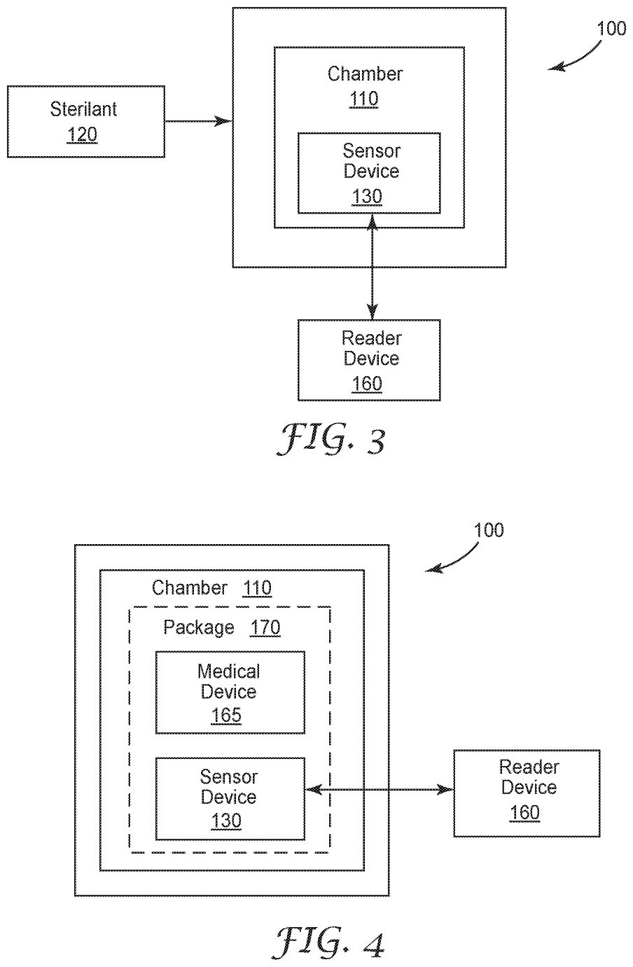

Referring now to FIG. 3, use of the sensor device 130 in sterilization system 100 in accordance with some embodiments of the present disclosure is illustrated. As shown, the sensor device 130 may be disposed within the chamber 110 of sterilization system 100. In some embodiments, the sensor device 130 may be disposed within the chamber 110 such that it may interact with the component(s) of the sterilant stream 120 upon entry into the chamber 110. In some embodiments, a reader device 160 may also be provided.

In some embodiments, the reader device 160 may be configured to receive signals from the sensor device 130 and translate the received signal into a determination that relates to the adequacy of a sterilization cycle (e.g., a pass/fail determination). For example, the reader device 160 may be configured to interrogate the sensor device 130 such that the reader device 160 measures the impedance across the electrode pair (e.g., induvial readings or continuous or semi-continuous readings over time) which can correspond to whether various environmental conditions were or were not achieved in the sterilization process, or whether an adequate sterilization process was achieved. In some embodiments, when exposed to a first environmental condition (e.g., ambient conditions), the reader device 160 (if interrogating the sensor device) would measure a first impedance value that is indicative of whether the conductive polymer of the electrical bridge 145 is in a first impedance state or a second impedance state. As described above, an environmental condition change (or second environmental condition) within the chamber 110 can change the impedance state of the conductive polymer and, in turn, the impedance across the electrode pair measured by the reader device 160. In some embodiments, when the conductive polymer is in the first impedance state a first resistance is measurable across the first and second electrode, and when the conductive polymer is in the second impedance state a second resistance is measurable across the first and second electrode, and the first resistance is different than the second resistance.

In some embodiments, the reader device 160 may be in electronic communication (or capable of electronic communication) (continuously or at any desired interval) with the sensor device 130 (e.g., wireless communication such as Bluetooth or RF communication or wired communication via a suitable electronic connection (e.g., a pair of electrical leads that may be coupled to an electrode pair of the sensor device 130)). In some embodiments, the reader device 160 may be a device for measuring electrical resistance (e.g., an electrical multimeter).

Referring now to FIG. 4, use of the sensor device 130 in a sterilization system 100 in accordance with some embodiments of the present disclosure is illustrated. As shown, the sensor device 130 may again be disposed within the chamber 110 of sterilization system 100 such that it may interact with the component(s) of the sterilant stream 120 upon entry into the chamber 110. Additionally, one or more medical devices 165 to be sterilized may be disposed with the chamber 110. For example, as shown, the sensor device 130 and the one or more medical devices 165 may be housed to together in a package 170 (often referred to in industry as a tray). It is to be appreciated that each package 170 may house any number of medical devices 165 or number of sensor devices 130. Alternatively, the sensor device 130 and the one or more medical devices 165 may be housed separately within the chamber 110. As shown, embodiments, a reader device 140 may also be provided.

In some embodiments, the present disclosure further relates to methods of using the sensor device 130 in a sterilization system 100. The method may begin with a user placing the sensor device 130 in the chamber 110. As previously discussed, the sensor device 130 may be placed alone in the chamber 110 or may be placed with one or more medical devices to be sterilized (and may be packaged in a tray with medical devices or disposed in the chamber 110 separate from the medical device or medical device tray). After the sensor device is placed in the chamber, the chamber 110 can be sealed from the environment.

In some embodiments, a user can then activate a sterilization process of the sterilizer and the sensor device can be exposed to a sterilant and/or one or more environmental conditions in a sterilization process. For example, if the sterilant is steam, then the sterilant may be at least 95% saturated steam/water vapor and the sterilization process may include achieving a temperature within the chamber 110 of at least 132 or at least 134 degrees Celsius for at least 2 minutes or at least 121 degrees Celsius for at least 8 minutes or at least 10 minutes. Various standards for each sterilant can exist and may vary based on the manufacturer, article to be sterilized, or combinations thereof.

In some embodiments, as discussed above, exposing the sensor 130 to the sterilant and/or the conditions within the chamber 110, may result in a change of the impedance state of the conductive polymer of the electrical bridge 145. For example, in embodiments in which the sterilant is steam, prior to sterilant exposure, because the latent base of the electrical bridge 145 is not soluble or poorly soluble in water at room temperature, the conductive polymer of the electrical bridge 145 may remain in its acid doped state. Exposing the electrical bridge 145 to steam, however, may cause the release of the latent base into the conductive polymer, thereby converting the conductive polymer to the dedoped, or second impedance state.

In some embodiments, the method may further include continuously, intermittently, or at any desired time, the reader device 160 receiving signals from the sensor device 130 and translating such received signal into a determination that relates to the adequacy of a sterilization cycle (e.g., a pass/fail determination). As discussed above, the received signals may relate to a measured impedance across the electrode pair, which corresponds to various environmental conditions that were or were not achieved in the sterilization process. For example, a measured impedance above or below a predetermined value may be used to determine whether adequate sterilization process conditions were achieved within the chamber 110.

### EXAMPLES

These examples are merely for illustrative purposes only and are not meant to be limiting on the scope of the appended claims. All parts, percentages, ratios, etc. in the examples and the rest of the specification are by weight, unless noted otherwise. Materials used in the examples and their sources are provided in Table 1. Solvents and other reagents used were obtained from the MilliporeSigma Company, St. Louis, MO, unless otherwise noted.

TABLE 1
Materials.

| Material | Source (Location) |
|---|---|
| Polyaniline (PANI) | Boron Molecular (Raleigh, NC) |
| Desmodur N3390 | Covestro LLC (Pittsburgh, PA) |
| CAPA 3031 | Perstorp Polyols Inc. (Toledo, OH) |
| Lithium carbonate fine powder | MilliporeSigma (St. Louis, MO) |
| Magnesium oxide nanoparticles (50 | MilliporeSigma (St. Louis, MO) |
| nm) | |
| Manganese carbonate fine powder | MilliporeSigma (St. Louis, MO) |
| Comply Bowie-Dick Test Pack | 3M Company (St Paul, MN) |
| Toluene | MilliporeSigma (St Louise, MO) |
| Methyl isobutyl ketone (MIBK) | MilliporeSigma (St Louise, MO) |
| Xylene | MilliporeSigma (St Louise, MO) |
| p-Tosylsulfonyl isocyanate | MilliporeSigma (St Louise, MO) |

### Example 1

The components provided in Table 2 were combined and mixed until homogeneous, producing a parent solution. Five grams of this parent solution was aliquoted to each of four vials. To three of the vials was added a latent base in the form of 50 mg of lithium carbonate powder, 50 mg magnesium oxide nanopowder (50 nm), or 50 mg of manganese carbonate fine powder. Each vial was sonicated for 20 minutes. These solutions each contained one percent of a latent base and may be referred to as base doped. All three bases are poorly soluble in aqueous solution. The fourth vial containing five grams of parent solution was used as a control.

TABLE 2
Parent solution.

| Component | Weight percent |
|---|---|
| 50% PANI in toluene | 14.92 |
| MIBK | 41.98 |
| Xylene | 29.57 |
| Desmodur 3390 | 8.48 |
| CAPA 3031 | 4.75 |
| p-Tosylsulfonyl isocyanate | 0.3 |

All solutions were mixed well with a small magnetic stirring bar for one hour before coating. Each solution was then coated on 3 mil polyethylene terephthalate (PET) film with a #16 Mayer bar and then dried and cured at 140° C. for 10 minutes. These coated PET films were then cut into approximately 5 cm by 10 cm strips. Two small squares (˜1 mm×˜1 mm) were drawn in the middle of each strip with a silver pen, with a gap of 1 cm between the two small squares. The silver paste was allowed to dry at room temperature for 5 minutes before measurement. The resistance of each coating was measured with a multimeter and the initial resistance values were recorded. These samples were subsequently steam sterilized by processing at 134° C. for 3.5 minutes with 4 vacuum pulses in an AMSCO Lab 110 steam sterilizer (Steris plc, Mentor, OH). Table 3 provides the color and electrical resistance of the samples before and after sterilization with steam.

As a control experiment, another set of samples was prepared and stored at more than 90% relative humidity (RH) and 50° C. for 3 hours (labeled “50° C./>90% RH” in Table 3) to examine the effect of moisture exposure under nonsterilizing conditions. Table 3 shows that both of the samples containing magnesium oxide and lithium carbonate showed very high resistances after the samples were exposed to steam sterilization, with the values more than 550 MΩ (which indicates overload of the multimeter). However, only lithium carbonate showed a clearly distinguished response between sterilization conditions and nonsterilizing moisture exposure, although the nonsterilizing moisture exposure sample did show a small increase in resistance to ˜20 MΩ.

TABLE 3
Electrical resistances and colors of base doped PANI coatings
before and after steam sterilization and moisture exposure.

| | | Resistance | Resistance | |
|---|---|---|---|---|
| | | before | after | |
| | | exposure | exposure | |
| Sample | Condition | (MΩ) | (MΩ) | Color |
| Control | | 0.6 | 0.6 | Green |
| Li₂CO₃ | No exposure | 5.74 | | Green |
| doped | 50° C./>90% RH | | 19.3 | Green |
| | Steam sterilization | | >550 | Blue |
| MnCO₃ | No exposure | 5.1 | | Green |
| doped | 50° C./>90% RH | | 15.3 | Green |
| | Steam sterilization | | 2.6 | Light Green |
| MgO | No exposure | 0.8 | | Green |
| doped | Steam | 0.8 | >550 | Light blue |
| | 50 C./>90% RH (3 h) | 0.8 | 478 | Blue |

### Example 2

A silver printed open circuit (Molex LLC, Naperville, IL) was coated with the 1% lithium carbonate doped parent solution prepared above using a #16 Mayer bar and cured at 140° C. for 4 minutes. This coated silver printed open circuit (similar in configuration to FIG. 2) was attached to a blotter paper card from a 3M Comply Bowie Dick test pack, providing a silver circuit card. The initial electric resistance of the coated silver printed open circuit was measured with a multimeter.

A COMPLY Bowie-Dick test pack (3M Company, St. Paul, MN) was carefully opened by cutting a slit along the tip of the wrapping paper located underneath the adhered label. This test pack comprises a pack or deck of blank blotter paper cards with a Bowie-Dick chemical indicator card in the middle of the blank blotter paper cards, all of which are wrapped with paper. The silver circuit cards were used to replace two of the original blotter paper cards in the opened Bowie-Dick test pack. One silver circuit card (card 1) replaced the fifth card ahead of the chemical indicator card and another silver circuit card (card 2) replaced the card immediately after the chemical indicator card. The chemical indicator card was maintained in its original position. This modified card stack was re-wrapped with the original wrapping paper and sealed with a small piece of SCOTCH tape (3M Company, St. Paul, MN) to close the slit.

This modified Bowie-Dick test pack was then subjected to a Bowie-Dick test cycle with a sterilization time of 3.5 minutes at 132° C. in an AMSCO Eagle 3013 steam sterilizer (Steris plc, Mentor, OH). After completion of the Bowie-Dick test cycle, the test pack was opened, the silver circuit cards were removed, and the conductivity of the attached circuits measured with a multimeter. Table 4 shows the electric resistance measurement results and colors observed. It can be seen that both cards showed changes in electrical resistance and in the color of the applied PANI coating upon steam sterilization. The results show that a silver circuit coated with a lithium carbonate doped PANI-containing coating can serve as an electric chemical indicator in an air removal test of a steam sterilizer.

TABLE 4
Electric resistance and color of coated electrodes wrapped into
a Bowie-Dick test pack before and after steam sterilization.

| | |
|---|---|
| Before sterilization | After sterilization |

| | Resistance | Color of | Resistance | Color of |
|---|---|---|---|---|
| Card # | (MΩ) | coating | (MΩ) | coating |
| 1 | 2.11 | Green | 21.3 | Spotted blue |
| 2 | 2.53 | Green | 9.37 | Spotted blue |

All references and publications cited herein are expressly incorporated herein by reference in their entirety into this disclosure. Illustrative embodiments of this disclosure are discussed and reference has been made to possible variations within the scope of this disclosure. For example, features depicted in connection with one illustrative embodiment may be used in connection with other embodiments of the disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below and equivalents thereof.

1. A sensor device comprising:
a first electrode and a second electrode, each of the first and second electrodes being electrically coupled to an electrical bridge;
the electrical bridge comprising:
a conductive polymer having a first impedance state and a second impedance state that is different than the first impedance state; and
a latent base.

2. The sensor device of claim 1, wherein the conductive polymer is configured to change from the first impedance state to the second impedance in response to a change in one or more environmental conditions.

3. The sensor device of claim 1, wherein the conductive polymer is configured to change from the first impedance state to the second impedance in response to exposure to an adequate environmental condition.

4. The sensor device of claim 1, wherein the latent base is configured to activate upon a change in one or more environmental conditions.

5. The sensor device of claim 1, wherein the latent base is configured to activate upon exposure to an adequate environmental condition.

6. The sensor device of claim 2, wherein the one or more changes in environmental condition comprise temperature and presence of a sterilant.

7. The sensor device of claim 6, wherein the sterilant comprises steam.

8. The sensor device of claim 1, wherein the latent base is disposed relative to the conductive polymer such that upon activation of the latent base the conductive polymer changes from the first impedance state to the second impedance state.

9. The sensor device of claim 1, wherein the first and second electrodes are electrically coupled to the electrical bridge such that when the conductive polymer is in the first impedance state, a first resistance is measurable across the first and second electrode, and when the when the conductive polymer is in the second impedance state a second resistance is measurable across the first and second electrode, and wherein the first resistance is different than the second resistance.

10. The sensor device of claim 1, wherein the conductive polymer comprises a repeat unit of: aniline, acetylene, pyrrole, phenylene, phenylene vinylene, phenylene ethynylene, phenylene sulfide, fluorene, pyrene, azulene, napthalene, carbazole, indol, thiophene, ethylene dioxythiophene, or combinations thereof.

11. The sensor device of claim 1, wherein the conductive polymer comprises polyaniline.

12. The sensor device of claim 7, wherein the polyaniline is acid-doped.

13. The sensor device of claim 1, wherein the latent base comprises a metal carbonate.

14. The sensor device of claim 1, wherein the electrical bridge further comprises a polymeric binder and wherein the conductive polymer and latent base are dispersed in the polymeric binder.

15. A sterilization system, the system comprising:
a sterilizer having a chamber configured to receive medical devices for sterilization; and
a sensor device of claim 1 disposed in the chamber.

16. A method, the method comprising:
providing a sensor device of claim 1;
exposing the sensor device to a sterilant in a sterilization process.

17. The method of claim 16, wherein the sterilant comprise at least 95% saturated steam and the adequate sterilization process is at least 132 degrees Celsius for at least 2 minutes or at least 121 degrees Celsius for at least 8 minutes.
